(19) United States
(12) Patent Application Publication
(10) Pub. No.: US 2003/0185911 A1
(43) Pub. Date: Oct. 2, 2003

(54) Herbal nutraceuticals and a process for preparing the same

(76) Inventors: Gulam Nabi Qazi, Jammu (IN); Chuni Lal Tickoo, Jammu (IN); Ashok Kumar Gupta, Jammu (IN); Devinder Kumar Gupta, Jammu (IN); Bupinder Singh Jaggi, Jammu (IN); Ram Pal Singh, Jammu (IN); Gurdeep Singh, Jammu (IN); Bal Krishan Chandan, Jimmu (IN); Krishan Avtar Suri, Jammu (IN); Naresh Kumar Satti, Jammu (IN); Vishwanath Gupta, Jammu (IN); Santosh Kumar Bakshi, Jammu (IN); Kasturi Lal Bedi, Jammu (IN); Om Parkash Suri, Jammu (IN); Satish Chander Puri, Jammu (IN); Preeti Somal, Jammu (IN); Surjit Singh, Jammu (IN); Anamika Khajuria, Jammu (IN); Sudesh Kumar Ganjoo, Jammu (IN); Arun Kumar, Jammu (IN)

Correspondence Address: SUGHRUE MION, PLLC, 2100 PENNSYLVANIA AVENUE, N.W., WASHINGTON, DC, 20037, US

(21) Appl. No.: 10/112,068

(22) Filed: Mar. 27, 2002

(51) Int. Cl. A61K 35/78

(52) U.S. Cl. 424/729; 424/725; 424/737

(57) ABSTRACT

The present invention relates to a novel herbal nutraceutical and more particularly to novel herbal jams and jellies with proven pharmacological activities such as memory enhancer, antioxidant, anti-depressant, immuno-modulator, adapatogen, health promoter and chemically standardized on the basis of one or more bioactive constituents and also to a process for the manufacture of said herbal nutraceuticals.

### FIELD OF THE INVENTION

The present invention relates to a novel herbal nutraceutical and more particularly to novel herbal jams and jellies with proven pharmacological activities such as memory enhancer, antioxidant, anti-depressant, immuno-modulator, adapatogen, health promoter and chemically standardized on the basis of one or more bioactive constituents. The present invention also relates to a process for the manufacture of said herbal nutraceuticals.

### BACKGROUND AND PRIOR ART TO THE INVENTION

Fruits and vegetables are among the most important foods of mankind as these are not only nutritive but are also sometimes indispensable for the maintenance of health. Preservation of fruits in one or the other form has been practiced in all parts of the world since time immemorial.

Among the preserved fruits, jams, jellies, marmalades etc. form an important class of products. During World War II, fairly large quantities of these were imported into India from USA, UK and Australia. Now a day, such products are being manufactured extensively in this country, as by-products in fruit canning units. They are also made in homes all over the country.

Lately, with regard to the invention and application of various chemicals in fresh fruits and food items, there is a increased concern about “green” technologies and products. It has led the public more interested in using medicines from natural sources. In addition to prescription drugs, there is also now great interest in food products, which in addition to providing nutrition, foster specific therapeutic effects. Until recently, biosciences regarded foods as chemically mundane, thus of little salience to disease process. During the earlier history of biomedicine, new products which combined pharmacological and dietetic treatments, were produced. Thus, the interest in the health potential of foods was renewed. This started a secular initiative from the advocates of holistic health” and from individuals from the health care system. By now the concept of health promoting foods is well established in the biomedical and nutrition sciences. This has resulted in growing of literature on “functional foods” and nutraceuticals. The term functional food is used to describe foods with added ingredients, being capable to producing certain health benefits. The term nutraceutical is used to describe a food or food component considered to provide medicinal and/or health benefits. These foods assist in the prevention or treatment of disease. [Debrovner, D (1992), American Druggist, 295, 36-40; Goldbug, I (1994) in “Functional foods, designer foods, pharma foods, nutraceuticals. Chapman and Hall, New York 544 PP; anonymous (1995) Journal of American Dietetic Association, 95, 493-496; Johns, T and Romeo, J. T. (1998) in “Functionality of food phytochemicals, Plenum press, New York, 261 pp]. According to Richard Firshein D. O. an expert in nutritional research and medicine, many of the ailments, prevalent today including fatigue, asthma, diabetes and cancer can be prevented or treated with nutraceuticals. Intense research efforts are under way to develop marketable products. There is a strong interest among European consumers in foods claiming health benefits. There are differences in the health claims that interest people in different European countries. The U.K. places great emphasis on claims related to heart disease and the German consumer is more interested in claims relating to general disease resistance. Nearly all-multinational food producers have such products in their line of goods or in development. These products are enriched with dietary fiber (57%), calcium (36%), vitamin C (35%) vitamin E (22%), iron (3%) and magnesium (3%).

There are some herbal food supplements available in the developed nations and a few in the developing ones. These include herbal teas, vitacel, pastas, diabetic foods and chyavanprash etc.

Herbal jam referred in the literature is strictly prepared as per the Charaka Samihita and is known as chyavanprash. This is known for its rejuvenative and health promotion effects. Other herbal jams cited in the literature contain powdered herbs such as Withania somnifera, Centella asiatica, Tinospora cordifolia, Viola odorata, Echinacea angustifolia, Ocimum sanctum etc.

Therefore, the products available in the market have following disadvantages.

1. Most of Herbal Jams/Jellies available in the market are Jams incorporated with spices i.e., Ginger Jam, spicy blue berry jam, Grapefruit Jelly, Garlic jelly etc. their main emphasis is on taste health benefits.

2. Herbal teas are not familiar to the common people and the products are not available in the developing countries. These are more often rejected on the basis of undesired taste.

3. The Chyavanprash a food-supplement for strength and energy is used only as a casual food supplement.

4. The jams containing herbs do not contain standardized herbs based on genuine markers present in them.

### OBJECTS OF THE PRESENT INVENTION

The main object of the present invention is to provide a novel herbal nutraceutical and more particularly to novel herbal jams and jellies with proven pharmacological activities such as memory enhancer, antioxidant, anti-depressant, immuno-modulator, adapatogen, health promoter and chemically standardized on the basis of one or more bioactive constituents.

The main object of the present invention is to provide a develop for commercial production, a range of delicious Jams/jellies which can be used by all ages with specific medicinal properties based on specific marker compounds available in the extracts of the herbal drugs.

Another object of the present invention is to provide a simple economical process for the production of a nutraceutical jams/jellies which contain herb(s) or their standardized extracts on the basis of one or more bio-active components and are combined in one convenient but tasty formula of fruits in the form of jams or jellies.

Yet another object of the present invention is to provide a composition of herbal jams and jellies, the purpose of which is to produce the desired therapeutic benefit to the consumer in addition to the health benefits associated with commonly used jams & jellies.

Still another object of the present invention is to formulate a product which can be spooned on to crumpets, scans, home cooked bread or even straight out of the jar, while maintaining elegance in taste, texture and flavor.

A further object of the present invention is to produce a product which is helpful in defending the body against the effects of our stressful environment and providing vitality and energy.

Another object of the present invention is to produce a product at affordable price.

Yet another object of the present invention is to formulate a product with global market acceptability.

### DETAILED DESCRIPTION OF THE PRESENT INVENTION

Accordingly, the present invention provides a novel herbal nutraceutical composition comprising 1 to 6 wt % of an extract or powdered plant parts of one or more herbs selected from the group Withania somnifera, Centella asiatica, Tinospora cordifolia, Viola odorata, Echinacea angustifolia and Ocimum sanctum, 12 to 40 wt % of edible fruits, 60-75 wt % of sugar, 1 to 2 wt % of a gelling agent and optionally, 1.5 to 3.0 wt % of food additives.

In an embodiment of the present invention, said composition comprises 1 to 5.3 wt % of an extract or powdered plant parts of one or more herbs selected from the group Withania somnifera, Centella asiatica, Tinospora cordifolia, Viola odorata, Echinacea angustifolia and Ocimum sanctum, 13.5 to 38.75 wt % of edible fruits, 70 wt % of sugar, 1.3 wt % of a gelling agent and optionally, 0.7 wt % of a favoring and coloring agents.

In another embodiment of the present invention, the nutraceutical composition may be Jam or Jelly.

In still another embodiment of the present invention, 1 to 2 wt % of the extract of the herbs is used in the composition.

In yet another embodiment of the present invention, the plant parts used for preparing the extract may be selected from the group comprising of aerial parts, roots and leaves.

In a further embodiment of the present invention, the extraction of the plant parts is effected using the solvents selected from the group comprising water, acidulated water, ethyl alcohol, acetone and ethyl acetate or their mixture thereof.

In one more embodiment of the present invention, the herbal extract is concentrated before use.

In one another embodiment of the present invention, the extract of the herb(s) is concentrated under evacuated condition to such a consistency which may contain active constituents or their marker constituents in such a concentration or dose so as to show therapeutic effect on the consumer.

In an embodiment of the present invention, the active ingredient/component/marker components remain in such a concentrate so as to recommend approximate dosage of 15 gm/day of the jam or jellies for consumption to show the adequate therapeutical effect on the consumer.

In another embodiment of the present invention, the extract of the herb(s) is concentrated under reduced pressure at 35-60° C. for incorporation in nutraceuticals.

In still another embodiment of the present invention, 3 to 6 wt % of powdered plants are used in the composition.

In a further embodiment of the present invention, the plant parts are ground fine using conventional means for use in the composition.

In one more embodiment of the present invention, the tender parts of the plants are used for powdering.

In one another embodiment of the present invention, the tender plant parts are dried in shade before they are powdered.

In an embodiment of the present invention, the tender plant parts are ground to pass through a 350 mesh.

In another embodiment of the present invention, the edible fruits may be selected from the group comprising of apple, mango, apricot, banana, pear and pine apple.

In still another embodiment of the present invention, the fruits are ripened and is used in the form of pulp.

In yet another embodiment of the present invention, the fruits are washed, peeled, cored and pitted before use.

In a further embodiment of the present invention, the gelling agent is high methoxyl pectin.

In one more embodiment of the present invention, the food additives are conventional flavoring agents, preservatives and coloring agents.

In one another embodiment of the present invention, the conventional flavoring agents are selected from alphonso mango essence, apple flavor, Rose oil and gerinol.

In an embodiment of the present invention, 1 to 2 wt % of conventional preservative is optionally added to the composition.

In another embodiment of the present invention, 1.2 to 1.5 wt % of citric acid is added to the composition.

In still another embodiment of the present invention, the coloring agent is edible. In yet another embodiment of the present invention, the coloring agent is selected from the group comprising of erythrosine and sunset yellow.

The present invention also provides a process for the manufacture of a herbal nutraceuticals such as Jams and Jellies, said process comprising the steps:

(a) preparing a pulp of edible fruits;

(b) powdering or preparing a concentrated extract of one or more herbs selected from the group comprising of Withania somnifera, Centella asiatica, Tinospora cordifolia, Viola odorata ,Echinacea angustifolia and Ocimum sanctum;

(c) mixing 1 to 6 wt % of the powdered plant parts or the extract with 12 to 40 wt % of fruit pulp;

(d) mixing 60 to 75 wt % of sugar and 1 to 2 wt % of high methoxyl pectin to the mixture of step (c);

(e) concentrating the mixture obtained from step (d) by conventional techniques till the mixture attains a concentration of 65° brix at temperatures less than 60° C.;

(f) adding acidulate to the aforesaid mixture and concentrating the contents to 68.0° brix under 15-50 torr at temperatures less than 60° C., and

(g) boiling the above-obtained mixture and optionally adding 1.5 to 3 wt % of food additives.

In an embodiment of the present invention, 1 to 6 wt % an extract or powdered plant part of one or more herbs selected from the group Withania somnifera, Centella asiatica, Tinospora cordifolia, Viola odorata, Echinacea angustifolia and Ocimum sanctum is mixed with 12 to 40 wt % of ripened edible fruits, 60-75 wt % of sugar, 1 to 2 wt % of a gelling agent and optionally, 1.5 to 3.0 wt % of food additives.

In another embodiment of the present invention, the ripened fruits are washed, peeled, cored and pitted before they are pulped.

In still another embodiment of the present invention, the fruits are washed with 0.2% aqueous hydrochloric acid followed by water washing.

In yet another embodiment of the present invention, the pulping is done using conventional techniques.

In a further embodiment of the present invention, the edible fruits may be selected from the group comprising of apple, mango, apricot, pear and pine apple.

In one more embodiment of the present invention, wherein in step (ii) the plant parts used for preparing the extract may be selected from the group comprising of aerial parts, roots and leaves.

In one another embodiment of the present invention, wherein in step (ii) the extraction of herb(s) is effected using the solvents selected from the group comprising water, acidulated water, ethyl alcohol, acetone and ethyl acetate or their mixture thereof.

In an embodiment of the present invention, the extract is centrifuged before use.

In another embodiment of the present invention, the herbal extract is concentrated before use.

In still another embodiment of the present invention, the extract of the herb(s) is concentrated under evacuated condition to such a consistency which may contain active constituents or their marker constituents in such a concentration or dose so as to show theraupeutic effect on the consumer.

In yet another embodiment of the present invention, the extract of the herb(s) is concentrated under reduced pressure at 35-60° C. for incorporation in nutraceuticals.

In a further embodiment of the present invention, the removal of excess water is effected by heating the mixture under reduced pressure at temperature 45±5° C.

In one more embodiment of the present invention, the active ingredient/component/marker components remain in such a concentrate so as to recommend approximate dosage of 15 gm/day of the jam or jellies for consumption to show the adequate therapeutical effect on the consumer.

In one another embodiment of the present invention, 1 to 2 wt % of the concentrated extract is used for preparing the composition.

In an embodiment of the present invention, the fine powdered plant parts are used for preparing the composition.

In another embodiment of the present invention, tender plant parts are ground fine using conventional techniques for use in the composition.

In still another embodiment of the present invention, plant parts dried in shade before they are ground.

In a further embodiment of the present invention, the plant parts are ground to pass through a 350 mesh.

In one more embodiment of the present invention, mixing of sugar and high methoxyl pectin is accompanied with constant stirring.

In one another embodiment of the present invention, wherein in step (e) the mixture is concentrated by evaporating the water at 55-65° C.

In an embodiment of the present invention, the evaporation is carried out under vacuum.

In another embodiment of the present invention, the food additives are conventional preservatives, flavoring agents and coloring agents.

In still another embodiment of the present invention, the conventional flavoring agents are selected from alphonso mango essence, apple flavor, Rose oil and gerinol are added to the composition.

In yet another embodiment of the present invention, 1 to 2 wt % of conventional preservative is optionally added to the composition.

In a further embodiment of the present invention, 1.2 to 1.5 wt % of citric acid is added to the composition.

In one more embodiment of the present invention, the coloring agent is edible.

In one another embodiment of the present invention, the coloring agent is selected from the group comprising of erythrosine and sunset yellow.

The present invention a process for the preparation of nutraceuticals and more particularly for herbal jams and jellies from the fruit pulp which comprises:

a) Procuring and sorting of fresh, mature and well ripe fruits.

b) Peeling, coring and pitting of the selected fruit.

c) Washing the fruit with 0.2% aqueous hydrochloric acid followed by water washing.

d) Pulping of the fruits.

e) Extraction of herb/herbs with water.

f) Centrifugation of the aqueous herbal extract

g) Concentration of the herbal extract under vacuum to the consistency of the fruit pulp

h) Mixing of the herbal extract with fruit pulp in a pan.

i) Addition of sugar and high methoxyl pectin to the mixture by stirring.

j) Evaporation of the water at 55-60° C. under vacuum till the concentration of the material is approximately 65° Brix. such that the therapeutically active ingredients of the herb remain in the desired concentration to show their effect in the product when consumed by the consumers.

k) Addition of acidulant to the mixture and concentration of the material to 68.50° Brix under vacuum.

l) Boiling of the contents and addition of coloring and flavoring matter by thorough mixing.

m) Filling of the finished product into the sterilized containers.

In a preferred embodiment of the present invention, said method comprises:

i. Washing the fruit with water or 0.2-0.4% aqueous hydrochloric acid.

iii. Pulping of the fruits by the use of pulping machine or by other methods.

iii. Extraction of herb/herbs with water, alcohol, acidulated water, acetone or any other organic solvent.

iv. Clarification of the herbal extract by centrifugation or by filtration.

v. Concentration of the extract under vacuum at 40±5° C. to the consistency of the fruit pulp

vi. Mixing of the concentrated or as such herbal extract with fruit pulp in a pan.

vii. Addition of sugar and high methoxyl pectin to the mixture by stirring at ambient temperature to 45° C.

viii. Evaporation of the water at room temperature to 45° C. under reduced pressure till the concentration of the material is approximately 60-65° Brix, such that the therapeutically active ingredients of the herb remain in the desired concentration to show their effect in the product when consumed by the consumers.

ix. Addition of acidulant to the mixture and concentration of the material to 67-70° Brix under 15-50 torr at a temperature 40±5° C.

x. Boiling of the contents and addition of vegetative or natural coloring and flavoring matter by thorough mixing.

The nutraceutical prepared by the present invention has the following characteristics:

1. Attractive color,

2. Well set,

3. Original fruit flavor,

4. Very good spreadability,

5. Very pleasant taste,

6. No separation of crystals at 10° C., and

7. No mould growth and fermentation at 37° C. for 7 days

The present invention is described in more detail in the following examples which are by the way of illustration only and therefore should not be construed to limit the scope of the invention.

### EXAMPLE-1

To the finely powdered (350 mesh) roots of Withania somnifera (7.2 gm) added 45 gm fresh mango pulp followed by the addition of 140 gm sugar by stirring at room temperature. The contents kept on burning gas stove, as soon as boiling set in, high methoxyl pectin (2.6 gm) added while stirring the contents. To the boiling mixture added 1.3 gm citric acid. Boiling continued till the desired concentration of the material (T.S.S. 68.5° Brix) is reached and finally 0.01 gm edible colour and 0.08 ml (alphanso mango) essence added after removing the burner. The finished product (200 gms) packed immediately in sterilized container. On bio evaluation, the product exhibited increase in cerebral resistance to oxygen by 35.88%, increase in physical endurance by 40.16% and anti fatigue by 50% as compared to vehicle treated animals.

### EXAMPLE-2

To the fresh apple pulp (36 gm) added 9 gm finally powdered (350 mesh) roots of Withania somnifera and Tinospora cordifolia (1:1) in a pan and mixed the ingredients thoroughly. To this added 140 gm powdered sugar and mixed it thoroughly on a heating burner. High methoxyl pectin (2.6 gm) added as soon as boiling sets in. To the boiling material 1.3 gm citric acid was added with stirring and concentration of the contents continued till it reached to 68° Brix. After removing the heating source 0.08 ml essence (apple flavour) and 0.01 gm edible colour added by mixing the contents thoroughly. The product (200 gm) packed into sterilized container while hot. Evaluation of the product exhibited 38.53%, 34.01% and 40.0% increase in survival under hypoxia, swimming endurance and antifatigue effect respectively.

### EXAMPLE-3

To the dried powdered and sieved (350 mesh) aerial parts of Ocimum sanctun, Centella asiatica and Echinacea angustifolia (1:1:1) 10.6 gm. mixed 27 gm fresh apple pulp followed by the mixing of 140 gm powdered sugar in a pan. The pan heated on a heater and added 1.3 gm of benzoic acid, High methoxyl pectin (2.6 gm) added by stirring and boiling of the is reached to 65° Brix. Continued boiling of the material upto 68-70° Brix concentration. After the addition of apple essence (0.0 8 ml) and erythrosine colour (0.01 gm) the contents (200 gm) packed in to the sterilized container while hot. Antistress activity of the product was observed by 37.86% increase in survival under hypoxia, (41.47%) swimming and (50%) antifatigue activity.

### EXAMPLE-4

To the concentrated alcoholic extract of Ocimum sanctum leaves (2 g dried extract) added 77.5 gm mango pulp and 140 gm sugar. The contents mixed thoroughly, and boiled and to it was added 2.6 gm high methoxyl pectin followed by the addition of 1.3 gm of citric acid by stirring, after the concentration of the material is approximately 65° Brix. Concentration of the contents continued till it reaches to 68-70° Brix. After removal of heating source 0.01 gm sunset yellow and 0.08 ml alphanso mango essence added by stirring which afforded 200 gm herbal jam. The nutraceutical showed increased survival under hypoxia by 49.56%, swimming increase in endurance by 58.27% and antifatigue after 30 minutes of stress 60%.

### EXAMPLE-5

Finally powdered aerial parts of Centella asiatica, Ocimum sanctum Echinacea angustifolia and roots of Withania somnifera (1:1:1:1) (100 gm) refluxed on water both for 4 hour with 1000 ml water. The contents filtered and marc again extracted with 900 ml water for 4 hour on boiling water both and filtered. The combined filtrate centrifuged and concentrated in a thin film evaporator, its solid residue was calculated by drying measured volume of concentrated extract.

To the aqueous extract (equivalent to 4 gm dry weight) added 65 gm of mango pulp and 140 gm of powdered sugar. The contents boiled and added 2.6 gm high methoxyl pectin. The material mixed thoroughly and distilled the water under reduced pressure at 50° C. till the concentration of the material is approx. 65° Brix. To the mixture then added 1.3 gm of citric acid and concentration of the material continued till the consistency of the material is 68.5° Brix. To the mixture, added 0.01 gm sunset yellow and 0.08 ml essence (alphanso mango) and mixed it thoroughly. The final product (200 gm) packed in sterilized container while hot. Treatment with product in animals showed increase in anti fatigue effect by 70%, swimming endurance by 53.60% and survival under hypoxia by 56.30%.

### EXAMPLE-6

Clarified, concentrated hot alcoholic extracts of Withania somnifera roots, Centella asiatica aerial parts, Echinacea angustifolia aerial parts & Ocimum sanctum leaves (mixed in equal quantity) equivalent to 4 gm of dry contents mixed with 65 gm of apple pomace and 140 gm powdered sugar. The contents boiled for five minutes and added to it high methoxyl pectin (2.6 gm). The mixture concentrated at 55° C. under reduced pressure till it attains the concentration of 65° Brix approximately. To this added citric acid (1.3 gm) and concentration of the material under diminished pressure continued upto 68.5° Brix. To this added erythrosine (0.008 gm) and apple flavor 0.1 ml and mixed it thoroughly. The final product 200 gm packed in sterilized container while hot. Effect of the nutraceutical exhibited in survival time under hypoxia by 53.78%, swimming endurance by 55.32% and anti fatigue by 80% after 7 days treatment.

### EXAMPLE-7

To the clarified, concentrated hot alcoholic extract of Tinospora cordifolia (equivalent to 4 gm of dry contents) added 65 gm rose marc and 140 gm powdered sugar, mixed the contents thoroughly and boiled it for five minutes. To this added 2.6 gm of high methoxyl pectin and concentrated it under reduced pressure by keeping the temperature of water bath at 50° C., to the concentration of approximately 65° Brix. To the contents added 1.3 gm of citric acid and concentration of the material continued in vacuum till 68.0° Brix. Finally, the product mixed with erythrosine (0.008 gm) and 0.1 ml essence (Mixture of rose oil and gerinol 1:3) and packed the final product (200 gm) in sterilized container. The product showed 29.43% increase in survival time under hypoxia, whereas increase in swimming performance 37.29% and anti fatigue 30.0% was observed.

### EXAMPLE-8

Centella asiatica (aerial parts 7.6 gm) after washing shade drying and powdering (350 mesh) mixed with 45gms of fresh mango pulp and 140 gm of powdered sugar. The mixed material boiled for five minutes on a heater and added to it high methoxyl pectin (2.6 gm). The mixture further boiled by stirring till it attains the concentration of approximately 65° Brix. To the contents than added 1.3 gm of citric acid by mixing and continued boiling up to 68.5° Brix. To the product finally added essence (alphanso mango flavor) 0.01 gm. and packed the green natural colored final product (200 gm) while hot in sterilized container. The product exhibited anti stress activity as observed by increase in survival time under hypoxia (34.78%), swimming performance (27.37) and anti fatigue after 30 minutes of the stress (40.0%)

The Main Advantages of the Present Process are:

1. Production of a product preferably jams or jellies which can be used by all age groups to revitalize both body and mind and provide both medicinal and health benefits.

2. Production of a nutraceutical which contain herbs of medicinal repute specially mentioned in Indian System of Medicines.

3. To produce a product which is relished by all ages of consumers and at the same time has curative as well as health promoting effect.

4. To produce a product which is easily marketable and affordable by even lower income groups.

1. A novel herbal nutraceutical composition comprising 1 to 6 wt % of an extract or powdered plant parts of one or more herbs selected from the group Withania somnifera, Centella asiatica, Tinospora cordifolia, Viola odorata, Echinacea angustifolia and Ocimum sanctum, 12 to 40 wt % of edible fruits, 60-75 wt % of sugar, 1 to 2 wt % of a gelling agent and optionally, 1.5 to 3.0 wt % of food additives.

2. A novel herbal nutraceutical composition as claimed in claim 1, wherein said composition comprises 1 to 5.3 wt % of an extract or powdered plant parts of one or more herbs selected from the group Withania somnifera, Centella asiatica, Tinospora cordifolia, Viola odorata, Echinacea angustifolia and Ocimum sanctum, 13.5 to 38.75 wt % of edible fruits, 70 wt % of sugar, 1.3 wt % of a gelling agent and optionally, 0.7 wt % of a favoring and coloring agents.

3. A herbal nutraceutical composition as claimed in claim 1, wherein the nutraceutical composition may be Jam or Jelly.

4. A herbal nutraceutical composition as claimed in claim 1, wherein 1 to 2 wt % of the extract of the herbs is used in the composition.

5. A herbal nutraceutical composition as claimed in claim 4, wherein the plant parts used for preparing the extract may be selected from the group comprising of aerial parts, roots and leaves.

6. A herbal nutraceutical composition as claimed in claim 4, wherein the extraction of the plant parts is effected using the solvents selected from the group comprising water, acidulated water, ethyl alcohol, acetone and ethyl acetate or their mixture thereof.

7. A herbal nutraceutical composition as claimed in claim 4, wherein the herbal extract is concentrated before use.

8. A herbal nutraceutical composition as claimed in claim 7, wherein the extract of the herb(s) is concentrated under evacuated condition to such a consistency which may contain active constituents or their marker constituents in such a concentration or dose so as to show therapeutic effect on the consumer.

9. A herbal nutraceutical composition as claimed in claim 8, wherein the active ingredient/component/marker components remain in such a concentrate so as to recommend approximate dosage of 15 gm/day of the jam or jellies for consumption to show the adequate therapeutical effect on the consumer.

10. A herbal nutraceutical composition as claimed in claim 7, wherein the extract of the herb(s) is concentrated under reduced pressure at 35-60° C. for incorporation in nutraceuticals.

11. A herbal nutraceutical composition as claimed in claim 1, wherein 3 to 6 wt % of powdered plants are used in the composition.

12. A herbal nutraceutical composition as claimed in claim 11, wherein the plant parts are ground fine using conventional means for use in the composition.

13. A herbal nutraceutical composition as claimed in claim 11, wherein the tender parts of the plants are used for powdering.

14. A herbal nutraceutical composition as claimed in claim 13, wherein the tender plant parts are dried in shade before they are powdered.

15. A herbal nutraceutical composition as claimed in claim 13, wherein the tender plant parts are ground to pass through a 350 mesh.

16. A herbal nutraceutical composition as claimed in claim 1, wherein the edible fruits may be selected from the group comprising of apple, mango, apricot, banana, pear and pine apple.

17. A herbal nutraceutical composition as claimed in claim 1, wherein the fruits are ripened and is used in the form of pulp.

18. A herbal nutraceutical composition as claimed in claim 1, wherein the fruits are washed, peeled, cored and pitted before use.

19. A herbal nutraceutical composition as claimed in claim 1, wherein the gelling agent is high methoxyl pectin.

20. A herbal nutraceutical composition as claimed in claim 1, wherein the food additives are conventional flavoring agents, preservatives and coloring agents.

21. A herbal composition as claimed in claim 20, wherein the conventional flavoring agents are selected from alphonso mango essence, apple flavor, Rose oil and gerinol.

22. A herbal nutraceutical composition as claimed in claim 20, wherein 1 to 2 wt % of conventional preservative is optionally added to the composition.

23. A herbal nutraceutical composition as claimed in claim 21, wherein 1.2 to 1.5 wt % of citric acid is added to the composition.

24. A herbal nutraceutical composition as claimed in claim 1, wherein the coloring agent is edible.

25. A herbal nutraceutical composition as claimed in claim 24, wherein the coloring agent is selected from the group comprising of erythrosine and sunset yellow.

26. A process for the manufacture of a herbal nutraceuticals such as Jams and Jellies, said process comprising the steps:
(a) preparing a pulp of edible fruits;
(b) powdering or preparing a concentrated extract of one or more herbs selected from the group comprising of Withania somnifera, Centella asiatica, Tinospora cordifolia, Viola odorata ,Echinacea angustifolia and Ocimum sanctum;
(c) mixing 1 to 6 wt % of the powdered plant parts or the extract with 12 to 40 wt % of fruit pulp;
(d) mixing 60 to 75 wt % of sugar and 1 to 2 wt % of high methoxyl pectin to the mixture of step (c);
(e) concentrating the mixture obtained from step (d) by conventional techniques till the mixture attains a concentration of 65° brix at temperatures less than 60° C.;
(f) adding acidulate to the aforesaid mixture and concentrating the contents to 68.0° brix under 15-50 torr at temperatures less than 60° C., and
(g) boiling the above-obtained mixture and optionally adding 1.5 to 3 wt % of food additives.

27. A process as claimed in claim 26, wherein 1 to 6 wt % an extract or powdered plant part of one or more herbs selected from the group Withania somnifera, Centella asiatica, Tinospora cordifolia, Viola odorata, Echinacea angustifolia and Ocimum sanctum is mixed with 12 to 40 wt % of ripened edible fruits, 60-75 wt % of sugar, 1 to 2 wt % of a gelling agent and optionally, 1.5 to 3.0 wt % of food additives.

28. A process as claimed in claim 26, wherein the ripened fruits are washed, peeled, cored and pitted before they are pulped.

29. A process as claimed in claim 28, wherein the fruits are washed with 0.2% aqueous hydrochloric acid followed by water washing.

30. A process as claimed in claim 26, wherein the pulping is done using conventional techniques.

31. A process as claimed in claim 26, wherein the edible fruits may be selected from the group comprising of apple, mango, apricot, pear and pine apple.

32. A process as claimed in claim 26, wherein in step (ii) the plant parts used for preparing the extract may be selected from the group comprising of aerial parts, roots and leaves.

33. A process as claimed in claim 26, wherein in step (ii) the extraction of herb(s) is effected using the solvents selected from the group comprising water, acidulated water, ethyl alcohol, acetone and ethyl acetate or their mixture thereof.

34. A process as claimed in claim 26, wherein the extract is centrifuged before use.

35. A process as claimed in claim 26, wherein the herbal extract is concentrated before use.

36. A process as claimed in claim 35, wherein the extract of the herb(s) is concentrated under evacuated condition to such a consistency which may contain active constituents or their marker constituents in such a concentration or dose so as to show theraupeutic effect on the consumer.

37. A process as claimed in claim 33, wherein the extract of the herb(s) is concentrated under reduced pressure at 35-60° C. for incorporation in nutraceuticals.

38. A process as claimed in claim 37, wherein the removal of excess water is effected by heating the mixture under reduced pressure at temperature 45±5° C.

39. A process as claimed in claim 26, wherein the active ingredient/component/marker components remain in such a concentrate so as to recommend approximate dosage of 15 gm/day of the jam or jellies for consumption to show the adequate therapeutical effect on the consumer.

40. A process as claimed in claim 36, wherein 1 to 2 wt % of the concentrated extract is used for preparing the composition.

41. A process as claimed in claim 26, wherein the fine powdered plant parts are used for preparing the composition.

42. A process as claimed in claim 26, wherein tender plant parts are ground fine using conventional techniques for use in the composition.

43. A process as claimed in claim 26, wherein plant parts dried in shade before they are ground.

44. A process as claimed in claim 26, wherein the plant parts are ground to pass through a 350 mesh.

45. A process as claimed in claim 26, wherein mixing of sugar and high methoxyl pectin is accompanied with constant stirring.

46. A process as claimed in claim 26, wherein in step (e) the mixture is concentrated by evaporating the water at 55-65° C.

47. A process as claimed in claim 46, wherein the evaporation is carried out under vacuum.

48. A process as claimed in claim 26, wherein the food additives are conventional preservatives, flavoring agents and coloring agents.

49. A process as claimed in claim 26, wherein conventional flavoring agents are selected from alphonso mango essence, apple flavor, Rose oil and gerinol are added to the composition.

50. A process as claimed in claim 26, wherein 1 to 2 wt % of conventional preservative is optionally added to the composition.

51. A process as claimed in claim 50, wherein 1.2 to 1.5 wt % of citric acid is added to the composition.

52. A process as claimed in claim 26, wherein the coloring agent is edible.

53. A process as claimed in claim 52, wherein the coloring agent is selected from the group comprising of erythrosine and sunset yellow.
